SEQUENCE LISTING (1 sequence)

SEQ ID NO: 1 - DNA, 4059 bp, synthetic construct
  source 1..4059: mol_type = other DNA
atgatccactccgtgtttctgctgatgttcctgctgacccctaccgagagctatgtggatgtgggccctgatagcgtgaagtccgcctgtatcgaagtggacatccagcagacctttttcgacaagacctggcctcggcctatcgacgtgtccaaggccgacggcatcatctaccctcaaggccggacctacagcaacatcaccattacctaccagggactgttcccctaccaaggcgaccacggcgatatgtacgtgtactctgctggacacgccaccggcaccacacctcagaaactgttcgtggccaactacagccaggacgtgaagcagttcgccaatggctttgttgtgcggatcggcgctgccgccaatagcacaggcacagtgatcatcagccctagcaccagcgccaccatcagaaagatctaccctgccttcatgctgggcagcagcgtgggcaatttctccgatggcaagatgggcagattcttcaatcacaccctggtgctgctgcctgatggctgtggaacactgctgcgggccttctactgcatcctggaacctagaagcggcaatcactgccctgccggcaacagctacaccagctttgccacataccacacaccagccacagactgcagcgacggcaactacaacagaaacgccagcctgaacagcttcaaagagtacttcaacctgcggaactgcaccttcatgtacacctacaatatcaccgaggacgagatcctcgagtggttcggcattacacagacagctcagggcgtgcacctgttcagcagcagatatgtggacctgtacggcggcaacatgttccagtttgccactctgcccgtgtacgacaccatcaagtactacagcatcatccctcacagcatccggtccatccagagcgatagaaaagcctgggccgccttctacgtgtacaagctgcagcctctgaccttcctgctggatttcagcgtggacggctacatcagaagggccatcgactgcggcttcaacgatctgagccagctgcactgctcctacgagagctttgatgtggaaagcggcgtgtactccgtgtctagctttgaggccaagcctagcggctctgtggttgaacaggctgaaggcgtggaatgcgacttctctcctctgctgtctggcacccctcctcaggtgtacaacttcaagcggctggtgttcaccaactgcaattacaacctgaccaagctgctgagcctgttctccgtgaacgacttcacctgtagccagatctctccagccgccattgccagcaactgctacagctccctgatcctggactacttcagctaccctctgagcatgaagtctgacctgagcgtgtcctctgccgggcctatcagccagtttaactacaagcagagcttcagcaaccccacctgtctgattctggccaccgtgcctcacaatctgaccaccatcaccaagcctctgaagtacagctacatcaacaagtgcagcagactgctgagcgacgacagaacagaagtgccccagctggtcaacgccaaccagtacagcccttgtgtgtccatcgtgcctagcacagtgtgggaggacggcgattactacagaaagcagctgagccctctggaaggcggaggatggcttgtggcttctggaagcacagtggccatgaccgaacagctgcagatgggctttggcatcaccgtgcagtacggcaccgataccaatagcgtgtgccctaagctggaattcgccaacgacaccaagatcgccagccagctgggaaactgcgtggaatactctctctacggcgtgtccggaagaggcgtgttccagaattgcacagccgtgggcgtccgccagcagagatttgtgtacgacgcctaccagaacctcgtgggctactacagcgacgatggcaactactactgcctgagagcctgtgtgtccgtgccagtgtccgtgatctacgacaaagagacaaagacccacgccacactgtttggcagcgtggcctgtgaacacatcagctccaccatgagccagtactccaggtccaccagatccatgctgaagcggagagacagcacatacggccctctgcagacacctgtgggatgtgtgctgggcctcgtgaacagcagcctgtttgtggaagattgcaagctgcctctgggccagtctctgtgtgctctgccagatacacctagcacactgaccccaagatctgtcagatccgtgcctggcgaaatgaggctggcctctatcgcctttaatcaccccatccaggtggaccagctgaactccagctacttcaagctgagcatccccaccaacttcagcttcggcgtgacccaagagtacatccagaccacaatccagaaagtgaccgtggactgcaagcagtacgtgtgcaacggctttcagaagtgcgagcagctcctgagagagtacggccagttctgcagcaagatcaatcaggccctgcacggcgccaacctgagacaggatgattccgtgcggaatctgttcgcctccgtgaagtctagccagagcagccctatcatccctggcttcggcggcgactttaatctgaccctgctggaaccagtgtccatcagcaccggctctagaagcgccagatctgccatcgaggacctgctgttcgacaaagtgaccattgccgatcctggctacatgcagggctacgacgattgcatgcagcagggacctgctagcgccagagatctgatttgtgcccagtacgtggccggctataaggtgctgcctcctctgatggacgtgaacatggaagccgcctacacaagcagcctgctgggatctattgccggcgttggatggacagccggactgagcagctttgctgccattcctttcgctcagagcatcttctaccgcctgaatggcgtgggcatcacccagcaggttctgagcgagaatcagaagctgatcgccaacaagttcaaccaggctctgggcgccatgcagaccggctttacaaccaccaacgaggccttcagaaaggtgcaggacgccgtgaacaacaacgcccaggcactgtctaaactggccagcgagctgagcaatacctttggcgccatctctgccagcatcggcgacatcattcagcggctggatgtgctggaacaggacgcccagatcgacagactgatcaacggcagactgaccacactgaacgcctttgtggcccagcagctcgtcagaagtgaatctgcagcactgtccgctcagctggccaaggacaaagtcaatgagtgcgtgaaggcccagagcaagagatccggcttttgtggccagggcacccacatcgtgtccttcgtggttaatgcccctaacggcctgtactttatgcacgtgggctattaccccagcaaccacatcgaggtggtgtccgcctatggcctgtgtgatgccgctaatcctaccaactgtatcgcccctgtgaacggctacttcatcaagaccaacaacacccggatcgtggacgagtggtcttacaccggcagcagcttttacgcccctgagcctatcacctctctgaacaccaaatacgtggcccctcaagtgacataccagaacatctccaccaacctgcctccacctctgctgggcaattccaccggcatcgactttcaggacgagctggacgagttcttcaagaacgtgtccaccagcattcccaacttcggcagcctgacacagatcaacaccacactgctggacctgacctacgagatgctgtccctgcagcaggtcgtgaaagccctgaacgagagctacatcgacctgaaagagctggggaactacacctactacaacaagtggccctggtacatctggctgggctttatcgctggactggtggctctggccctgtgcgtgttcttcatcctgtgttgtaccggctgcggcaccaattgcatgggcaagctgaaatgcaaccggtgctgcgacagatacgaggaatacgatctggaaccccacaaggtgcacgtccac

(19) United States
(12) Patent Application Publication
(10) Pub. No.: US 2026/0069675 A1
(43) Pub. Date: Mar. 12, 2026

(54) NUCLEIC ACID VACCINES ENCAPSULATED WITH NANOALUMINOSILICATE AGAINST MIDDLE EAST RESPIRATORY SYNDROME CORONAVIRUS (MERS-CoV)

(71) Applicant: Imam Abdulrahman Bin Faisal University, Dammam (SA)

(72) Inventors: Iman Almansour ALZAMIL, Dammam (SA); Rabindran JERMY, Dammam (SA)

(73) Assignee: Imam Abdulrahman Bin Faisal University, Dammam (SA)

(21) Appl. No.: 18/883,134

(22) Filed: Sep. 12, 2024

(51) Int. Cl. A61K 39/215; A61K 9/00; A61K 9/51; A61K 39/00; A61P 31/14; C12N 7/00; C12N 15/70

(52) U.S. Cl. CPC A61K 39/215; A61K 9/0019; A61K 9/5115; A61P 31/14; C12N 7/00; C12N 15/70; A61K 2039/53; A61K 2039/552; A61K 2039/55505; A61K 2039/575; C12N 2770/20034; C12N 2770/20051

(57) ABSTRACT

A MERS-CoV vaccine with nucleic acid sequences having at least 90% identity to the spike gene of a MERS-CoV strain as a preventive measure against MERS-CoV infections is described. The nucleic acid sequences may include plasmid DNA (pDNA) or messenger RNA (mRNA) that are encapsulated by aluminosilicates. The aluminosilicates may be functionalized with aminopropyltrimethoxysilanes.

### STATEMENT REGARDING PRIOR DISCLOSURE BY THE INVENTORS

Aspects of the present disclosure are described in Almansour, I. and Jermy, B. R., “Nucleic acid vaccine candidates encapsulated with mesoporous silica nanoparticles against MERS-CoV” published in Issue 1, Volume 20, 2346390, Human Vaccines & Immunotherapeutics, which is incorporated herein by reference in its entirety.

### STATEMENT OF ACKNOWLEDGEMENT

Support provided by Deputyship for Research and Innovation Ministry of Education in Saudi Arabia through project number 2017-131-IRMC is gratefully acknowledged.

### REFERENCE TO SEQUENCE LISTING

In accordance with 37 CFR § 1.52 (e) (5) and with 37 CFR § 1.831, the specification makes reference to a Sequence Listing submitted electronically as a .xml file named “551860US Sequence Listing”. The .xml file was generated on Sep. 4, 2024, and is 5,701 bytes in size. The entire contents of the Sequence Listing are hereby incorporated by reference.

### BACKGROUND

### Technical Field

The present disclosure is directed to nucleic acid vaccines against Middle East Respiratory Syndrome Coronavirus (MERS-CoV), wherein the vaccines comprise plasmid DNA (pDNA) or messenger RNA (mRNA) encapsulated in a nanoaluminosilicate.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

Middle east respiratory coronavirus (MERS-CoV) is a respiratory viral disease that belongs to the Orthocoronaviridae subfamily of Coronaviridae. The Orthocoronaviridae are positive sense, single-stranded RNA that consists of 4 genera: alpha-CoV, beta-CoV, gamma-CoV, and delta-CoV. Severe acute respiratory syndrome coronavirus-1 (SARS-CoV-1), SARS-CoV-2, and MERS-CoV all belong to the beta-CoV genus; however, infection with MERS-CoV is associated with a higher mortality rate compared to SARS-CoV-2. The MERS-CoV genome is about 30 kilobase (Kb) that consists of 11 open reading frames (ORF) [Woo, P. C., Lau, S. K., Huang, Y., & Yuen, K. Y., Coronavirus diversity, phylogeny and interspecies jumping. Experimental Biology and medicine, 2009, 234 (10), 1117-1127). S gene, in particular, is a class I transmembrane glycoprotein. This protein can be cleaved into two non-covalently bond units: an N-terminal globular S1 subunit and a membrane-proximal S2 subunit. The S1 contains the receptor binding domain (RBD) responsible for virus attachment to the host cell receptor, while the S2 contains the fusion domain, thereby mediating the virus entry [Wang, Q., Wong, G., Lu, G., Yan, J., & Gao, G. F. (2016). MERS-CoV spike protein: targets for vaccines and therapeutics. Antiviral research, 2016, 133, 165-177]. Unlike SARS-CoV and SARS-CoV-2 where an angiotensin-converting enzyme 2 (ACE-2) is used for virus cellular entry, MERS-CoV is dependent on dipeptidyl peptidase 4 (DPP-4 or CD26) for its entry into a host cell [Raj, V. S., et al., Dipeptidyl peptidase 4 is a functional receptor for the emerging human coronavirus-EMC. Nature, 2013, 495 (7440), 251-254; and Kindler, E., et al., Efficient replication of the novel human betacoronavirus EMC on primary human epithelium highlights its zoonotic potential. MBio, 2013, 4 (1), 10-1128]. The S protein is the primary antigenic determinant in coronaviruses that elicits antibody-mediated responses against the virus. Humoral antibody responses have been shown to protect infected animals and humans.

The MERS-CoV case was first reported in June 2012 and has been associated with ongoing sporadic outbreaks since then [Ramadan, N., & Shaib, H., Middle East respiratory syndrome coronavirus (MERS-CoV): A review. Germs, 2019, 9 (1), 35; and Mertz, G. J., and Wormser, G. P., Zoonoses: infectious diseases transmissible from animals to humans, fourth edition, Clin Infect Dis., 2016, 63 (1): 148.2-9]. Since then, MERS-CoV infected more than 2500 people globally, including more than 800 deaths. Due to its high fatality rate (˜40%), MERS-CoV is categorized as one of the high-priority pathogens identified by the World Health Organization (WHO). Currently, no human vaccines against MERS-CoV has been approved. The S protein is the primary target for the immune response generated toward MERS-CoV; therefore, efforts made to develop vaccine candidates against MERS-CoV have focused on the S protein. MERS-CoV vaccine candidates have been explored preclinically and clinically, including live attenuated, viral vector, and pDNA. Nucleic acid-based vaccines, including pDNA and mRNA vaccines, promise next-generation vaccine platforms [Gary, E. N., & Weiner, D. B., DNA vaccines: prime time is now. Current Opinion in Immunology, 2020, 65, 21-27]. While conventional vaccines are typically manufactured as bulk production of vaccines in mammalian cells or egg embryos, nucleic acid-based vaccines use host cell machinery to produce the encoded protein antigen. pDNA vaccines, in particular, offer a number of advantages compared to conventional vaccines, including ease of production, cost-effectiveness, and high stability [Liu, M. A., A comparison of plasmid DNA and mRNA as vaccine technologies. Vaccines, 2019, 7 (2), 37.]. Contrastingly, mRNA is inherently unstable and involves encapsulation to protect mRNA from physical degradation by RNAse. Furthermore, the delivery of mRNA into host cells requires efficient intracellular delivery [Liu, M. A., A comparison of plasmid DNA and mRNA as vaccine technologies. Vaccines, 2019, 7 (2), 37; and Ho, W., Gao, M., Li, F., Li, Z., Zhang, X. Q., & Xu, X. Next-generation vaccines: nanoparticle-mediated DNA and mRNA delivery. Advanced Healthcare Materials, 2021, 10 (8), 2001812].

Several nanoparticles encapsulating mRNA have been tested and reported as promising technologies with low genotoxicity and high biocompatibility. As such, lipid-based nanoparticles have reached clinical trial application and subsequent approval for mRNA-based COVID-19 vaccines [Anselmo A C, Mitragotri S. Nanoparticles in the clinic: An update post COVID-19 vaccines. Bioengineering & translational medicine. 2021 6 (3): e10246]. In addition, graphene quantum dots (GQD) functionalized with polyethyleneimine polymer have been used in RNA-based therapeutics. the cationic charge density of the polymer has been shown to improve the RNA complexation and effective delivery to liver cells [Liu, Y., Zhao, C., Sabirsh, A., Ye, L., Wu, X., Lu, H., and Liu, J., A Novel Graphene Quantum Dot-Based mRNA Delivery Platform. ChemistryOpen. 2021]. Further, a dendric mesoporous/ZIF-8 nanocomposite has been used in mRNA delivery. The presence of mesopores and tetrasulfide bonds on the pore walls assisting nanocrystals of ZIF-8 was reported to influence high mRNA loading and transfection efficacy [Wang, Y., Song, H., Liu, C., Zhang, Y., Kong, Y., Tang, J., Yang, Y., and Yu, C., Confined growth of ZIF-8 in dendritic mesoporous organosilica nanoparticles as bioregulators for enhanced mRNA delivery in vivo. National Science Review. 2021, (8): 268].

Application of lipid nanocarriers for mRNA delivery has garned much attention, with less attention being paid to inorganic aluminosilicate nanomaterials. Due to its versatile features, nanoporous and/or mesoporous silica is a multifunctional platform that has been developed and tested in a wide range of vaccine and drug delivery systems, including pDNA and mRNA vaccines and therapeutics. Mesoporous silica with tunable pore diameters (2-50 nm) and large surface texture has been reported as a candidate for gene delivery (˜1000 m²/g) [Janjua, T. I., Cao, Y., Yu, C., Popat, A., Clinical translation of silica nanoparticles. Nature Reviews Materials. 2021, (12): 1072-4]. Mesoporous silica capped with template quaternary trimethyl ammonium as functionalized linker along with imidazolo-oxindole PKR inhibitor C16 has been used in the delivery of mRNA [Zhang, W., Liu, Y., Chin, J. M., Phua, K. K., Sustained release of PKR inhibitor C16 from mesoporous silica nanoparticles significantly enhances mRNA translation and anti-tumor vaccination. European Journal of Pharmaceutics and Biopharmaceutics. 2021, 163:179-87]. Structured silica with functional abilities and uniform particle size is suited for siRNA-mediated gene silencing [Hom, C., Lu, J., Liong, M., Luo, H., Li, Z., Zink, J. I., Tamanoi, F., Mesoporous Silica Nanoparticles Facilitate Delivery of siRNA to Shutdown Signaling Pathways in Mammalian Cells, Small. 2010, 6 (11): 1185-1190]. Silylation using amino functional moieties such as 3-aminopropyltriethoxysilane (APTES), aminopropyl trimethoxysilane (APTMS), and carbodiimide routes are strategies for loading RNA and subsequent release of genes into various applications [Zhou, Y., Quan, G., Wu, Q., Zhang, X., Niu, B., Wu, B., Huang, Y., Pan, X., Wu, C., Mesoporous silica nanoparticles for drug and gene delivery. Acta Pharmaceutica sinica B. 2018, 8 (2): 165-77].

Several types of nano silica have been reported for the delivery of nucleic acid-based vaccines. DNA may be encapsulated by an APTES functionalized spherical silica sample; however, it is difficult to encapsulate RNA, as RNA encapsulation requires a more positive charge than DNA encapsulation. Additionally, no previous study has explored mRNA as a potential vaccine candidate for MERS-CoV. While the surface glycoprotein of MERS-CoV is immunogenic, in-vitro synthesis of mRNA encoding S can be challenging.

Accordingly, an object of the present disclosure is to provide a nucleic acid-based vaccine, particularly an mRNA-based vaccine for MERS-CoV. Another object of the present disclosure is to provide a suitable nanocarrier based on aminopropyltrimethoxysilane (APTMOS) functionalized spherical silica for the delivery of nucleic acid vaccines.

### SUMMARY

In an exemplary embodiment, a Middle East Respiratory Syndrome coronavirus (MERS-CoV) vaccine is disclosed. The MERS-CoV vaccine includes a nanoparticle fusion. The nanoparticle fusion comprises an immunogenic composition and an aluminosilicate.

In some embodiments, the immunogenic composition comprises a nucleic acid molecule with a nucleic acid sequence having at least 90% identity over an entire length of the nucleic acid sequence from a spike(S) gene of a MERS-CoV strain, wherein the nucleic acid molecule is either in the form of a plasmid deoxyribonucleic acid (pDNA) or a messenger ribonucleic acid (mRNA).

In some embodiments, the aluminosilicate is functionalized with an aminopropyltrimethoxysilane.

In some embodiments, the nucleic acid molecule is encapsulated by the aminopropyltrimethoxysilane functionalized aluminosilicate.

In some embodiments, the aminopropyltrimethoxysilane is 3-aminopropyltrimethoxysilane.

In some embodiments, the pDNA is made by a process comprising cloning the nucleic acid sequence into a pVax1 vector to form a construct; transforming the construct into HB101 competent cells; incubating the cells in a lysogeny broth media containing kanamycin at 35 to 40° C. for 8 to 16 hours; and purifying the incubated cells to extract the pDNA.

In some embodiments, the method of cloning the nucleic acid sequence into the pVax1 vector includes splicing the pVax1 vector at a BamHI flanking site and a NheI flanking site.

In some embodiments, the mRNA is made by a process comprising preparing linearized DNA from the pDNA, wherein the linearized DNA contains a T7 promoter, and transcribing and capping the linearized DNA into the mRNA using an mMESSAGE transcription kit.

In some embodiments, a molar ratio of silicon dioxide to aluminum oxide in the aluminosilicate is from 70:1 to 90:1.

In some embodiments, the nucleic acid sequence has a length of 4000 to 4100 nucleotides.

In some embodiments, the nucleic acid sequence is SEQ ID No: 1.

In some embodiments, a weight ratio of the aluminosilicate to the nucleic acid sequence is from 100:1 to 200:1.

In some embodiments, an average guanine-cytosine content (GC-content) in the nucleic acid sequence is from 40 to 70% based on a total amount of base pairs in the nucleic acid sequence.

In some embodiments, the aminopropyltrimethoxysilane functionalized aluminosilicate has an average pore size distribution of 8 to 9 nm.

In some embodiments, the aminopropyltrimethoxysilane functionalized aluminosilicate has a Brunauer-Emmett-Teller (BET) surface area of 120 to 160 m²/g.

In some embodiments, the aminopropyltrimethoxysilane functionalized aluminosilicate has a Barrett, Joyner, Halenda (BJH) adsorption cumulative surface area of 80 to 90 m²/g.

In some embodiments, the aminopropyltrimethoxysilane functionalized aluminosilicate has a pore volume of 0.2 to 0.4 cm³/g.

In some embodiments, the nucleic acid molecule is pDNA, and the aminopropyltrimethoxysilane functionalized aluminosilicate has an encapsulation efficiency of 45 to 50% based on an initial amount of the pDNA.

In some embodiments, the nucleic acid molecule is mRNA, and the aminopropyltrimethoxysilane functionalized aluminosilicate has an encapsulation efficiency of 52 to 58% based on an initial amount of the mRNA.

In some embodiments, the nucleic acid molecule is pDNA, and the vaccine comprises the nucleic acid molecule in an amount of 80 to 120 μg.

In some embodiments, the nucleic acid molecule is mRNA, and the vaccine comprises the nucleic acid in an amount of 20 to 40 μg.

In some embodiments, a method of MERS-CoV prevention is disclosed. The method includes administering the vaccine through one or more intramuscular needle administrations to animal models to lower a risk of contracting MERS-CoV.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of the present disclosure (including alternatives and/or variations thereof) and many of the attendant advantages thereof will be readily obtained as the same becomes better understood by reference to the following detailed description of the embodiments when considered in connection with the accompanying drawings, wherein:

FIG. 1 illustrates a codon-optimized, full-length S DNA sequence of MERS-CoV, according to certain embodiments.

FIG. 2 is a flowchart depicting a process for the synthesis of plasmid DNA (pDNA), according to certain embodiments.

FIG. 3 is a flowchart depicting a process for the synthesis of messenger RNA (mRNA), according to certain embodiments.

FIG. 4 shows a full-length protein sequence of MERS-CoV spike(S) protein, according to certain embodiments.

FIG. 5A shows codon quality distribution of MERS-CoV S gene obtained by GeneOptimizer, according to certain embodiments.

FIG. 5B shows codon quality plot of MERS-CoV S gene obtained by GeneOptimizer, according to certain embodiments.

FIG. 5C shows the average GC contents in 40 bp window centered at an indicated nucleotide position, according to certain embodiments.

FIG. 6A shows agarose gel electrophoresis for two clones of pDNA expressing S gene, according to certain embodiments.

FIG. 6B shows agarose gel electrophoresis for in-vitro (IVT) synthesized mRNA, according to certain embodiments.

FIG. 7A shows the X-ray diffraction of HAS (A) and APTMOS-HAS (B), according to certain embodiments.

FIG. 7B shows the thermogravimetric analysis (TGA) of APTMOS-HAS (C and D), according to certain embodiments.

FIG. 7C shows the nitrogen adsorption isotherm of ZSM-5 (E), HAS (F), and APTMOS-HAS (G), according to certain embodiments.

FIG. 7D shows the pore size distribution of ZSM-5 (H), HAS (I), and APTMOS-HAS (J), according to certain embodiments.

FIG. 8A shows transmission electron microscope (TEM) images depicting morphology of HAS, according to certain embodiments.

FIG. 8B shows TEM images depicting the morphology of APTMOS-hierarchical AIMCM-41, according to certain embodiments.

FIG. 8C shows TEM images depicting the morphology of APTMOS-HAS at a scale of 50 nm, according to certain embodiments.

FIG. 8D shows TEM images depicting the morphology of APTMOS-HAS at a scale of 10 nm, according to certain embodiments.

FIG. 9 shows the 0.8% agarose gel analysis for the MERS-CoV vaccine candidates untreated, subjected to heat inactivation, and subjected to TURBO DNase digestion, according to certain embodiments.

FIG. 10A shows the humoral immune response in vaccinated mice groups against MERS-CoV S.FL measured by indirect ELISA at week 0, according to certain embodiments.

FIG. 10B shows the humoral immune response in vaccinated mice groups against MERS-CoV S.FL measured by indirect ELISA at week 2 according to certain embodiments.

FIG. 10C shows the humoral immune response in vaccinated mice groups against MERS-CoV S.FL measured by indirect ELISA at week 4, according to certain embodiments.

FIG. 10D shows the humoral immune response in vaccinated mice groups against MERS-CoV S.FL measured by indirect ELISA at week 6, according to certain embodiments.

FIG. 11 shows the binding antibodies responses in vaccinated groups elicited against MERS-CoV S1 and S.FL measured by indirect ELISA at week 6, according to certain embodiments.

### DETAILED DESCRIPTION

In the following description, it is understood that other embodiments may be utilized, and structural and operational changes may be made without departure from the scope of the present embodiments disclosed herein.

Reference will now be made in detail to specific embodiments or features, examples of which are illustrated in the accompanying drawings. Whenever possible, corresponding or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

In the drawings, like reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term “nucleic acid” refers to nucleotides and nucleosides which make up biomolecules, for example, DNA macromolecules and RNA macromolecules. A nucleic acid may be deoxyribonucleic acid (DNA) or ribonucleic acid (RNA). A nucleic acid may be single-stranded or double-stranded. Nucleic acids are biopolymers and macromolecules comprising nucleotides.

As used herein, the term “plasmid DNA (pDNA)” (also referred to as a plasmid) refers to a circular, double-stranded DNA molecule physically separate from chromosomal DNA. Plasmid DNA can replicate independently. Plasmid DNA typically have few numbers of genes (a sequence of nucleotides), which are able to be spliced and edited. Plasmid DNA can pass on the genes from one cell to another. Plasmid DNA may range in size from 1 to >200 kb. Plasmids occur naturally in bacteria and other microorganisms.

As used herein, the term “messenger RNA (mRNA)” refers to a single-stranded molecule of RNA that corresponds to a genetic sequence of a gene. Messenger RNA is an RNA molecule made from a DNA sequence by the process of transcription and is used for protein synthesis.

As used herein, the term “vector” refers to a DNA molecule (often a plasmid or a virus) that acts as a carrier to transfer a DNA sequence to a host cell.

As used herein, the term “protein” refers to a complex molecule comprised of one or more chains of amino acids (each called a polypeptide) serving in biological functions, such as structure, function, and regulation of cells, tissues, and organs.

As used herein, the term “codon” refers to a set of three nucleotides in nucleic acid molecules, DNA or RNA, that together code for an amino acid.

As used herein, the term “nucleotide” refers to monomeric units of nucleic acids, DNA and RNA, which are composed of a nitrogenous base, five-carbon sugar, and a phosphate group. The nitrogenous base, or nucleobase, may be adenine “A”, cytosine “C”, guanine “G”, thymine “T”, uracil “U”, and any nitrogenous base known in the art.

As used herein, the term “antigen” refers to a protein, peptide, lipid, moiety, toxin, foreign particulate matter, polysaccharide, allergen, nucleic acid, or any other similar molecule that can bind to a specific antibody or T-cell receptor and is capable of triggering an immune response in the body, particularly the production of antibodies.

As used herein, the term “antibody” refers to proteins produced by the body in response to and counteracting a specific antigen. Antibodies produce an immune reaction, particularly in response to a foreign antigen that has entered the body.

As used herein, the term “antigenic determinant” (also known as an epitope) refers to a part of the antigen recognized by various components of the immune system, including antibodies, B-cells, and T-cells.

As used herein, the term “immunogenic” refers to the ability of a foreign substance to generate an immune response in the body of a human or an animal.

As used herein, the term “immune response” refers to the physiological defense reaction of the immune system against the entry of a foreign substance or a microorganism, including bacteria and viruses.

As used herein, the term “humoral immune response” refers to the immune response mediated by antibodies produced by B-cells of the body in response to an attack by antigens. A humoral immune response is mediated by macromolecules, including secreted antibodies, complement proteins, and antimicrobial peptides, located in extracellular fluids.

As used herein, the term “vaccine” refers to a biological preparation that provides active acquired immunity to a particular or malignant disease. Herein, a vaccine is an antigenic composition that stimulates the immune system of the body and activates B-cells or T-cells to generate an immune response.

As used herein, the term “encapsulated nucleic acid” refers to a nucleic acid that is covered with a substance that protects the nucleic acid from degradation and also facilitates an efficient delivery of nucleic acid to the host.

As used herein, the term “specificity” refers to the ability of an antibody and/or an antigen to differentiate between other antibodies and/or antigens.

As used herein, the term “electrophoresis” refers to a technique to separate protein or nucleic acid molecules based on their size and electrical charge.

As used herein, the term “buffer” refers to a solution that can resist pH change after dilution or addition of small quantities of an acid or base.

As used herein, the term “pore size” may be considered as the lengths or longest dimensions of a pore opening.

As used herein, the term “pore volume” refers to the void volume of a porous structure and is calculated as void content per unit weight (cm³/g).

As used herein, the term “microporous” refers to materials having a pore diameter of less than 2 nm.

As used herein, the term “mesoporous” refers to materials having a pore diameter of about 2 nm to 50 nm.

As used herein, the term “hierarchical” refers to materials having both micropores and macropores. The material may have an inner area with micropores and an outer area with mesopores, and vice-versa. The material may have micropores and mesopores throughout.

Aspects of the present disclosure are directed to nucleic acid vaccines against Middle East Respiratory Syndrome coronavirus (MERS-CoV). More particularly, the present disclosure relates to nucleic acid vaccines comprising pDNA or mRNA encapsulated with 3-aminopropyltrimethoxysilane (APTMOS) functionalized hierarchical aluminosilicate (HAS), designated as APTMOS-HAS. pDNA vaccine technology is stable and is a highly flexible platform with the capability to induce both a humoral and cellular immune response without the need of further encapsulation step. This is also achieved through a robust, simple, and scalable GMP production. Contrastingly, mRNA is an intrinsically unstable molecule, making it a challenging platform. Various techniques have been explored to enhance mRNA stability, including 5′ capping, 3′ polyA tail length, and encapsulation. Further development of nanoparticles holds potential for retaining mRNA stability and for efficient intracellular delivery of mRNA vaccines and therapeutics. The selection of nanoparticles may enhance the efficiency of intracellular delivery and protection from nucleases.

Mesoporous silica nanoparticles are widely used in pharmaceutical formulations and have been proven to be safe in a wide array of pharmaceutical applications. As such, nano-silica has excellent biocompatibility and is chemically stable. Like lipid-based nanoparticles, nano-silica has been shown to play a role in antibody binding and cell interaction. mRNA and pDNA vaccine candidates against MERS-CoV were developed using the S gene. Further, a 3-aminopropyltrimethoxysilane (APTMOS) functionalized hierarchical aluminosilicate (HAS) was synthesized, designated as APTMOS-HAS, as a nanocarrier for mRNA and pDNA vaccine candidates. The mRNA vaccine candidate was developed against MERS-CoV, and APTMOS-HAS and was explored as a candidate for nucleic acid vaccine encapsulation. As such, a parallel immunological evaluation of non-encapsulated pDNA and mRNA vaccine candidates vs pDNA and mRNA vaccine candidates loaded in APTMOS functionalized spherical silica was performed. A slight improvement in immunogenicity after mRNA encapsulation was observed. Humoral immune response was improved after encapsulating mRNA. pDNA encapsulation did not show statistically significant immunogenicity enhancement. Contrastingly, the pDNA vaccine elicited the highest antibody response compared to mRNA alone or encapsulated mRNA.

The vaccine according to the present disclosure comprises an immunogenic composition wherein the immunogenic composition comprises a nucleic acid molecule. The nucleic acid molecule consists of a sequence derived from the spike(S) gene of Middle East Respiratory Coronavirus (MERS-CoV). The S gene is composed of an S1 subunit that contains the receptor binding domain for the attachment of the virus to the host cells and an S2 subunit that mediates the entry of the virus into the host. The S protein is a glycoprotein produced from the translation of the S gene and is responsible for the generation of the immune response against the virus in the host. All and/or parts of the spike gene can be used in the vaccines of the present disclosure. The nucleic acid sequence may include a naturally occurring sequence or variants of the naturally occurring sequence. In some embodiments, the vaccine comprises a nucleic acid sequence having at least 90% identity to the nucleic acid sequence encoding the spike gene of MERS-CoV strain. In some embodiments, the nucleic acid sequence may have at least 91%, at least 92%, at least 93%, at least 94%, at least 95%, at least 96%, at least 97%, at least 98%, at least 99%, or 100% identity to the nucleic acid sequence encoding the spike gene of MERS-CoV strain. In some embodiments, the nucleic acid sequence encoding the spike gene of MERS-CoV strain is preferably described by SEQ ID No. 1. In other embodiments, the nucleic acid sequence encoding the spike gene of MERS-CoV strain may be longer or shorter than that described by SEQ ID No. 1. In an embodiment, the nucleic acid sequence is SEQ ID No. 1.

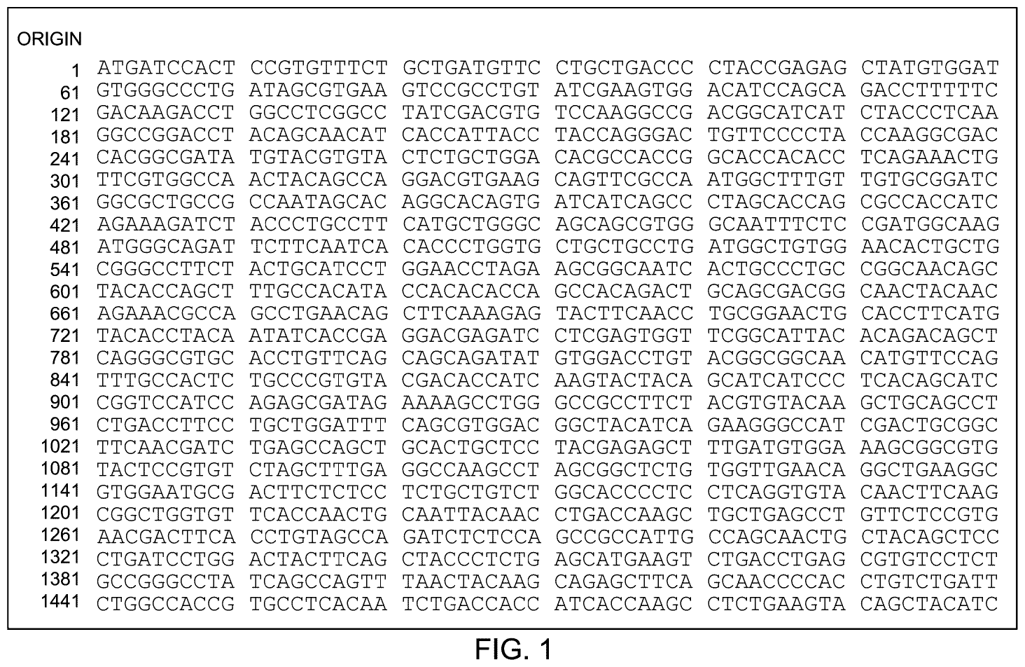

In some embodiments, the nucleic acid may include deoxyribonucleic acid (DNA). In certain embodiments, the nucleic acid comprises plasmid deoxyribonucleic acid (pDNA) synthesized from the nucleic acid sequence encoding the spike gene of MERS-CoV strain. In certain embodiments, the plasmid DNA is synthesized from the nucleic acid sequence, as provided in FIG. 1. In some embodiments, selection of codons is done during the synthesis of the plasmid DNA. Improvement of codons is preferred to enhance the gene expression in the host after immunization. In one embodiment, codons are identified by substituting synonymous codons for certain codons present in the spike gene. The synonymous codons are selected in a way that the protein sequence, obtained after the translation of the optimized gene, remains unaltered. In a preferred embodiment, the optimization of the spike gene is done for mammalian codons. In some embodiments, the noncoding regions of the nucleic acid sequence are removed to enhance the gene expression. The noncoding regions may comprise cis-acting sites, including splice donor and acceptor sites, promotor regions of genes, such as a TATA box and/or a CAAT box, RNA instability motif repeats, chi-sites, ribosomal entry sites, and the like.

In some embodiments, the nucleic acid sequence has 4000 to 4100 nucleotides. In certain embodiments, the nucleic acid sequence may have about 4010 to 4090 nucleotides, preferably about 4020 to 4080 nucleotides, more preferably about 4030 to 4070 nucleotides, and yet more preferably about 4040 to 4060 nucleotides. In a preferred embodiment, the nucleic acid sequence has about 4059 nucleotides.

In some embodiments, the nucleotide content in the nucleic acid sequence is adjusted to enhance the half-life of the nucleic acid. The content of any or all nucleotides, including adenine (A), guanine (G), thymine (T), cytosine (C), and uracil (U), may be varied if needed. The guanine (G)-cytosine (C) content determines the stability of a nucleic acid sequence. The guanine-cytosine (GC) content of the nucleic acid sequence is preferably altered to prolong the stability of the nucleic acid. In one embodiment, the GC content in the nucleic acid sequence is adjusted to 40 to 70% based on the total amount of base pairs in the nucleic acid sequence. In another embodiment, the GC content in the nucleic acid sequence is adjusted to 45 to 65%, preferably 50 to 60%, more preferably 54 to 58%, and yet more preferably about 56%, based on the total amount of base pairs in the nucleic acid sequence.

FIG. 2 illustrates a schematic flow diagram of the process 100 for the synthesis of plasmid DNA. The order in which process 100 is described is not intended to be construed as a limitation, and any number of the described steps can be combined in any order to implement process 100. Additionally, individual steps may be removed or skipped from the process 100 without departing from the spirit and scope of the present disclosure.

At step 102, the process 100 includes cloning the nucleic acid sequence of spike gene, as given in FIG. 1, into pVax1 to form a construct. In some embodiments, the S gene is first chemically synthesized and cloned into a vector. Any vector suitable for cloning genes for DNA-based vaccines may be used. The vector may include a plasmid, cosmid, bacteriophage, or a bacterial or yeast artificial chromosome. In some embodiments, the vector is an expression construct capable of delivering the desired gene into the host cell. In some embodiments, the vector is a circular plasmid. In other embodiments, the vector is a linear nucleic acid. The linear nucleic acid may be a linear DNA or may be derived from a plasmid that can be linearized. Suitable examples of vectors that can be used for cloning the spike gene include pVax, provax, pBR322, pUC19, pM2, or any other vector known in the art. In a preferred embodiment, the spike gene is cloned into a pVax1 vector.

Splicing sites for restriction enzymes may be introduced in the vector to cleave the DNA sequence for cloning. Restriction enzymes for which splicing sites may be introduced include EcoRI, EcoRII, BamHI, NheI, XbaI, HindIII, and the like. In certain embodiments, at least one flanking site for BamHI and at least one flanking site for NheI are added to the DNA sequence in the vector. In one embodiment, the flanking sites are added upstream to the DNA sequence in the vector. In another embodiment, the flanking sites are added downstream to the DNA sequence in the vector. In one more embodiment, the flanking sites are added upstream and downstream to the DNA sequence in the vector. In a preferred embodiment, the BamHI flanking sites are added downstream to the DNA sequence in the vector, and NheI flanking sites are added upstream to the DNA sequence in the vector. An S gene construct is obtained after cloning the sequence into pVax1.

At step 104, the process 100 includes transforming the construct into competent cells. Competent cells are derived from microorganisms that can take up the nucleic acid sequences from the surroundings through transformation. Bacterial or yeast cells can be used as competent cells, although bacterial cells are preferred because of their high transformation efficiencies. Competent cells suitable for the present disclosure include cells from E. coli strains such as DH5α, BL21, HB101, NEB5α, JM109, and the like. In some embodiments, the DNA construct is transformed into HB101-competent cells. The competency of the microbial cells is enhanced either chemically or by electroporation. Chemically competent cells may be given heat and chemical treatments to increase their transformation efficiency. The competency of cells can also be enhanced by giving electrical pulses to the cells that open up the cell membrane for DNA uptake. The construct may be transformed into any competent cells known in the art.

At step 106, the process 100 includes incubating the competent cells in a broth medium suitable for the growth of bacterial cells. Media rich in carbon sources, vitamins, minerals, peptones, and peptides are preferably used to cultivate the competent cells. In certain embodiments, lysogeny broth (LB) media grows and maintains the competent cells. LB media may be chosen as it enhances the growth rate of bacterial cells, increasing the overall yield of the plasmids. Any broth media known in the art may be used to grow the competent cells. Suitable antibiotics may be added to the broth media to grow the cells of antibiotic-resistant bacterial strains selectively. Antibiotics, including ampicillin, kanamycin, gentamycin, tetracyclin, chloramphenicol, bleocin, and the like, may be added to the broth. In some embodiments, ampicillin is added to the broth. In a preferred embodiment, kanamycin is added to the lysogeny broth to grow the competent cells.

The competent cells are incubated in the lysogeny broth at a temperature of 35 to 40° C. In some embodiments, the competent cells are incubated in the lysogeny broth at temperature of about 36 to 39° C., preferably 37 to 38° C. In a preferred embodiment, the competent cells are incubated in the lysogeny broth at about 37° C. The incubation is done for a period of 8 to 16 hours. In certain embodiments, the incubation is done for a period of about 9 to 15 hours, preferably about 10 to 14 hours, more preferably about 11 to 13 hours, and yet more preferably about 12 to 13 hours. In a preferred embodiment, the incubation of competent cells in the LB media is done for a period of about 12 hours.

At step 108, the process 100 includes purifying the incubated cells using a purification kit. Readily available purification kits can be used for the purification of the incubated cells. In certain embodiments, ChargeSwitch-Pro filter plasmid Maxiprep purification kit (Cat #CS31106, Thermo Fisher Scientific, USA) is used for the purification of incubated cells. The kit comprises a column for filtering the bacterial lysates and another column with a ChargeSwitch membrane designed to bind the plasmid DNA from the lysates. The purification is done in a single step without adding solvents, and purified plasmid DNA is extracted for use in the vaccine. Any purification kit known in the art may be used for purifying the incubated cells.

In some embodiments, the nucleic acid may include ribonucleic acid (RNA). In preferred embodiments, the nucleic acid may include a messenger ribonucleic acid (mRNA). The mRNA may be prepared from the pDNA synthesized from the nucleic acid sequence, as provided in FIG. 1. For RNA synthesis, any readily available kit for in-vitro synthesis may be used. In certain embodiments, mMessege mMachine T7 kit (Cat #AM1344, Thermo Fisher Scientific, Lithuania) is used for mRNA synthesis.

FIG. 3 illustrates a process 200 for synthesis of mRNA according to the present disclosure. The order in which process 200 is described is not intended to be construed as a limitation, and any number of the described steps can be combined in any order to implement process 200. Additionally, individual steps may be removed or skipped from the process 200 without departing from the spirit and scope of the present disclosure.

At step 202, the process 200 includes linearizing pDNA to obtain a linearized DNA. The pDNA is linearized to remove all the non-coding regions of the DNA to obtain high-quality mRNA in good quantities. The linearization of pDNA can be achieved by cleavage of the restriction sites by restriction enzymes. In certain embodiments, the pDNA is cleaved by the enzyme BamHI at its restriction site to obtain a linearized DNA.

The linearized DNA is transcribed into RNA. The process of transcription is mediated by polymerase enzymes that transcribe a DNA template into an RNA sequence. The polymerases are preferably DNA-dependent RNA polymerases. In one embodiment, the RNA polymerase is selected from a group consisting of T7 RNA polymerase, SP6 RNA polymerase, E. coli RNA polymerase, and T3 RNA polymerase. In some embodiments, the linearized DNA is transcribed by T3 RNA polymerase. In other embodiments, the linearized DNA is transcribed by SP6 RNA polymerase. In a preferred embodiment, the linearized DNA is transcribed by T7 RNA polymerase. The pDNA may be linearized by any polymerase enzyme known in the art.

In certain embodiments, a promoter sequence is inserted into the linearized DNA. The promoter sequence provides a binding site on the DNA sequence for the RNA polymerase enzyme to initiate transcription. In some embodiments, the promoter sequence may be placed upstream on the linearized DNA to allow transcription of the DNA downstream to the promoter sequence. The promoter sequence has a high binding affinity for the RNA polymerases and may be selected based on the type of RNA polymerase to be used for transcription. Accordingly, the promoter sequence may be selected for the T7 polymerase, the T3 polymerase, the SP6 polymerase, or any other polymerase known in the art. In one embodiment, the promoter sequence is for the T3 RNA polymerase. In another embodiment, the promoter sequence is for SP6 RNA polymerase. In a preferred embodiment, the promoter sequence is for T7 RNA polymerase.

At step 204, the process 200 includes transcription of the linearized DNA into mRNA by the enzyme RNA polymerase. Once the transcription is complete, the process 200 includes capping of the synthesized mRNA to prevent the degradation of the mRNA by other enzymes, such as phosphatases and nucleases. The process of capping includes addition of a 7-methyl guanosine cap at the 5′ end of the mRNA. The transcription and capping may be done using a readily available kit. In a specific embodiment, the transcription and capping of the synthesized mRNA is done using mMessege mMachine T7 kit (Cat #AM1344, Thermo Fisher Scientific, Lithuania). In other embodiments, the transcription and capping may be done with any methods and/or kits known in the art.

At step 206, the process 200 includes polyadenylation of the capped mRNA wherein multiple adenosine nucleotides are added to the mRNA to form a polyadenosine (polyA) tail. In one embodiment, the polyA tails comprises about 50 to 400 adenosine nucleotides. In another embodiment, the polyA tails comprises about 100 to 350 adenosine nucleotides, preferably 150 to 300 adenosine nucleotides, preferably 200 to 250 adenosine nucleotides. In a preferred embodiment, about 300 adenosine nucleotides are added to the 3′ terminus of mRNA. The polyA tail is added to the 3′ terminus of the mRNA wherein the polyA tail protects the 3′ end of the mRNA sequence from degradation by enzymes and provides a stop signal to the cell nucleus for further transcription. The polyA tail enables the transport of the synthesized mRNA from the nucleus to the cytoplasm of the cell for translation. The polyA tail may be derived from a DNA template by transcription or may be taken from polyadenylation kits. In specific embodiments, the polyadenylation of mRNA is carried out using a polyadenylation kit (Cat #AM1350, Thermo Fisher Scientific, Lithuania).

The polyadenylation of mRNA is followed by purification of the mRNA to remove nucleotides, short oligonucleotides, proteins, and salts. The purification of the mRNA can be done using purification kits. For example, the purification of mRNA may be done using MEGAclear transcription clean up kit (Cat #AM1908, Thermo Fisher Scientific, Lithuania). The purification may be done with any methods and/or kits known in the art.

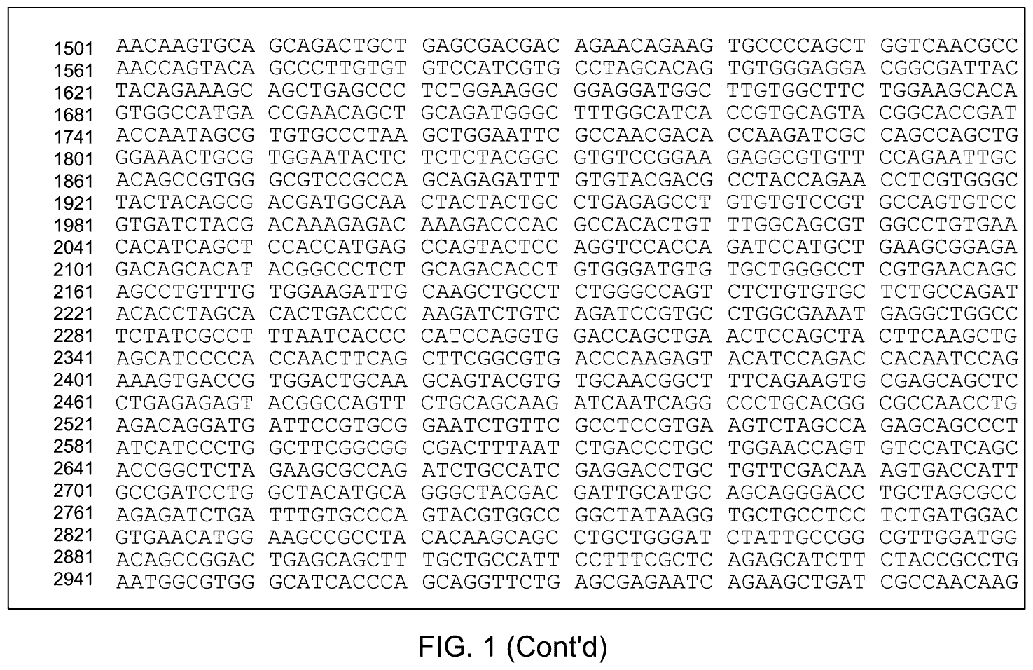

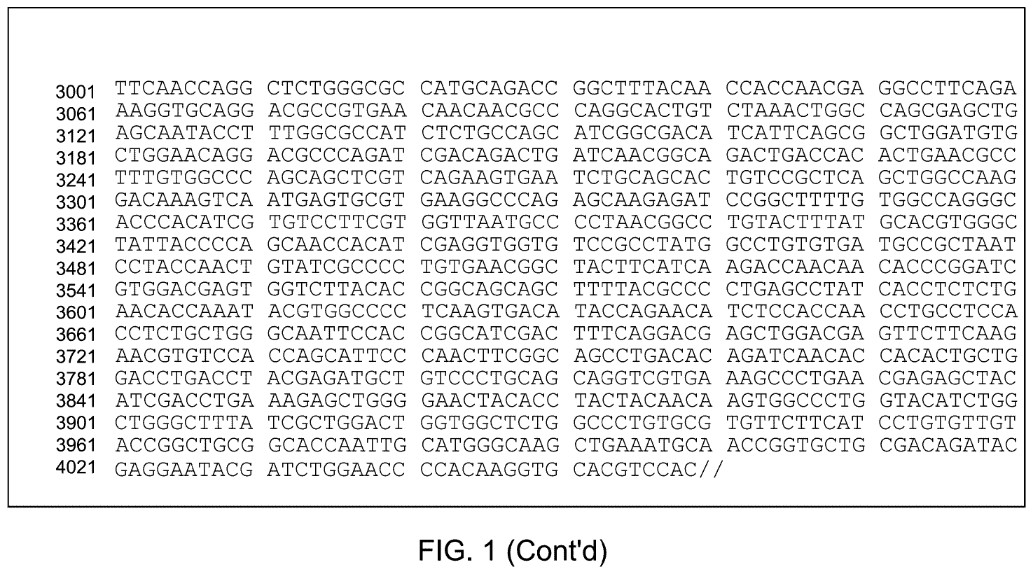

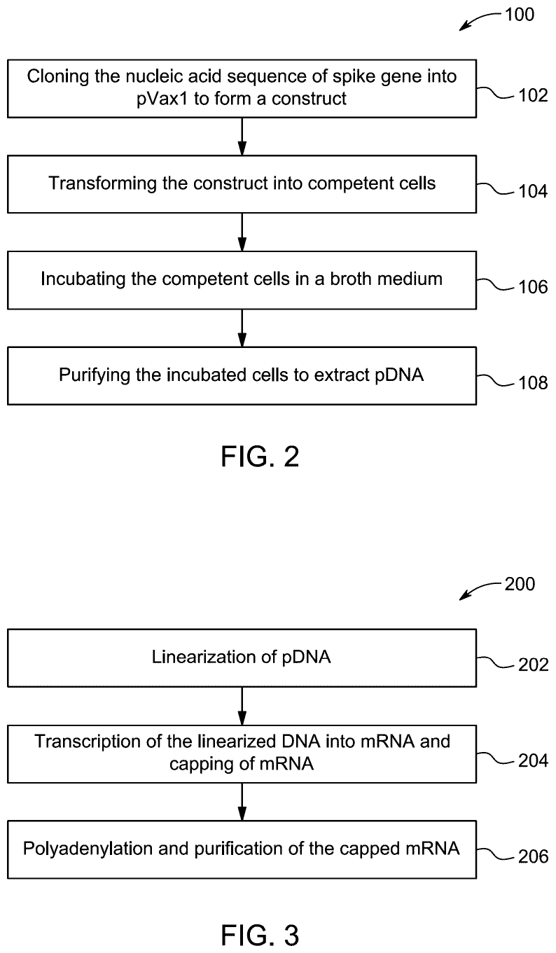

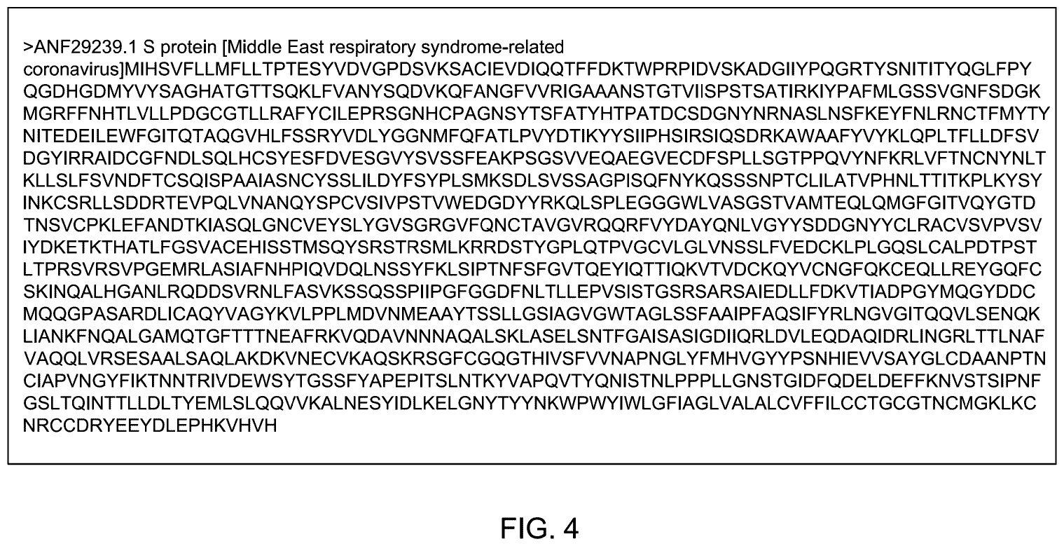

The purified mRNA is assessed for its quality by gel electrophoresis and visualized under UV using any non-radioactive marker. The synthesized mRNA may comprise the coding region for the full-length spike protein (FIG. 4) or for a fragment having the antigenic determinant for the spike protein. In one embodiment, the mRNA comprises at least one coding region comprised in the protein sequence depicted in FIG. 4.

In one embodiment, the MERS-CoV vaccine comprises a nanocarrier for efficient delivery of the immunogenic composition in the host cell. The nanocarrier encapsulates the immunogenic composition to prevent the composition from being degraded by the host cell's enzymes. In one embodiment, the nanocarrier is an aluminosilicate. The aluminosilicates contain Si—O—Al linkages and are composed of aluminum oxide and silicon dioxide. The encapsulation of nucleic acid may desire a higher positive charge on the surface of the nanocarrier. Accordingly, the aluminum is incorporated into silica which enhances the positive charge on the surface of silica, and the nucleic acid, particularly mRNA, is effectively encapsulated.

In certain embodiments, a hierarchical aluminosilicate (HAS) is prepared by introducing micro- and meso-hexagonal pores in the aluminosilicate structure. Hierarchical aluminosilicate (HAS) remains stable compared to other conventionally available mesoporous aluminosilicate and can provide extensive diffusional access to nucleic acid molecules. HAS possesses a high number of silanol groups relevant for surface functionalization and has the ability to modify to a mesopore form using several templates, such as CTAB, F127, and the like, to increase surface area support (up to 1100 m²/g) with a large pore volume (1 cc/g) and pore diameter (4 nm). The presence of such characteristics may be a beneficial platform to load the nucleic acid inside the large pores, while impregnating diagnostic nanoparticles such as oxide-based Au, Ti, and/or Fe on the large external surface area for dual applications of gene delivery.

The hierarchical aluminosilicate (HAS) may comprise silicon dioxide and aluminum oxide in a molar ratio of 70:1 to 90:1. In certain embodiments, the hierarchical aluminosilicate (HAS) may comprise silicon dioxide and aluminum oxide in a molar ratio of 72:1 to 88:1, preferably 74:1 to 86:1, preferably 76:1 to 84:1, more preferably 78:1 to 82:1, and yet more preferably about 80:1. In a preferred embodiment, the hierarchical aluminosilicate (HAS) may comprise a silicon dioxide and aluminum oxide in a molar ratio of 80:1.

In some embodiments, the aluminosilicate may be functionalized with a suitable silane group to increase the hydrophobicity on the encapsulated surface. Suitable silane groups that can be used include, but are not limited to, aminopropyltriethoxysilane, aminopropyltrimethoxysilane, and/or chloropropyl trimethoxysilane, and the like. In specific embodiments, the aluminosilicate may be functionalized with a suitable aminopropyltrimethoxysilane group (APTMOS). In a preferred embodiment, the aluminosilicate may be functionalized with 3-aminopropyltrimethoxysilane group.

In some embodiments, the aminopropyltrimethoxysilane functionalized aluminosilicate is layered. In some embodiments, the aminopropyltrimethoxysilane functionalized aluminosilicate is porous and has an average pore size distribution of 8 to 9 nm. In other embodiments, the aminopropyltrimethoxysilane functionalized aluminosilicate has an average pore size distribution of 8.1 to 8.9 nm, preferably 8.2 to 8.8 nm, preferably 8.3 to 8.7 nm, preferably 8.4 to 8.6 nm. In certain embodiments, the APTMOS functionalized aluminosilicate has an average pore size distribution of 8.2 to 8.5 nm. In one preferred embodiment, the APTMOS functionalized aluminosilicate has an average pore size distribution of about 8.2 nm.

In certain embodiments, the aminopropyltrimethoxysilane functionalized aluminosilicate is porous and has a pore volume of 0.2 to 0.4 cm³/g. In specific embodiments, the aminopropyltrimethoxysilane functionalized aluminosilicate has a pore volume of 0.21 to 0.39 cm³/g, preferably 0.22 to 0.38 cm³/g, preferably 0.23 to 0.37 cm³/g, preferably 0.24 to 0.36 cm³/g, preferably 0.25 to 0.35 cm³/g, preferably 0.26 to 0.34 cm³/g, preferably 0.27 to 0.33 cm³/g, preferably 0.28 to 0.32 cm³/g, preferably 0.29 to 0.31 cm³/g. In a preferred embodiment, the aminopropyltrimethoxysilane functionalized aluminosilicate has a pore volume of about 0.28 cm³/g.

The surface area of the APTMOS functionalized aluminosilicate as measured by Brunauer-Emmett-Teller (BET) technique is 120 to 160 m²/g. In certain embodiments, the BET surface area for the APTMOS functionalized aluminosilicate is in the range of 125 to 155 m²/g. In another embodiment, the BET surface area for the APTMOS functionalized aluminosilicate is in the range of 122 to 148 m²/g, preferably in the range of 124 to 146 m²/g, preferably 126 to 144 m²/g, preferably 128 to 142 m²/g, preferably 130 to 140 m²/g, more preferably 132 to 138 m²/g, and yet more preferably about 138 m²/g.

The adsorption cumulative surface area of the functionalized aluminosilicates is calculated based on an adsorption isotherm using the Barrett, Joyner, Halenda (BJH) technique. The APTMOS functionalized aluminosilicate is found to have an adsorption cumulative surface area of 80 to 90 m²/g, preferably 82 to 88 m²/g, more preferably 83 to 85 m²/g, and yet more preferably about 84 m²/g.

In one embodiment, the weight ratio of the aluminosilicate to the nucleic acid sequence may range from 100:1 to 200:1. In some embodiments, the weight ratio of the aluminosilicate to the nucleic acid sequence may range from 110:1 to 190:1, preferably 120:1 to 180:1, preferably 130:1 to 170:1, more preferably 140:1 to 160:1, and yet more preferably about 150:1.

In certain embodiments, the MERS-CoV vaccine comprises pDNA as the nucleic acid molecule. In other embodiments, the MERS-CoV vaccine comprises mRNA as the nucleic acid molecule. The encapsulation efficiency of the aminopropyltrimethoxysilane functionalized aluminosilicate varies depending on the type of nucleic acid in the immunogenic composition. The encapsulation with the aminopropyltrimethoxysilane functionalized aluminosilicate at least partially covers a surface area of the nucleic acid, e.g., preferably at least 50%, 70%, or 90% of the nucleic acid is covered with the aminopropyltrimethoxysilane functionalized aluminosilicate. The nucleic acid may be fully covered with the aminopropyltrimethoxysilane functionalized aluminosilicate. For example, the encapsulation efficiency of the aminopropyltrimethoxysilane functionalized aluminosilicate in cases where the immunogenic composition comprises pDNA is from 45 to 50%, preferably 46 to 49%, more preferably 47 to 48%, and yet more preferably about 47% based on an initial amount of pDNA. In some embodiments, the MERS-CoV vaccine comprises pDNA as the nucleic acid molecule and the nucleic acid is partially encapsulated by the aminopropyltrimethoxysilane functionalized aluminosilicate. In some embodiments, the MERS-CoV vaccine comprises pDNA as the nucleic acid molecule and the nucleic acid is at least 50%, preferably at least 60%, preferably at least 70%, preferably at least 80%, more preferably at least 90%, and yet more preferably at least 95% of the nucleic acid is covered by the aminopropyltrimethoxysilane functionalized aluminosilicate. In other embodiments, where the immunogenic composition comprises mRNA as the nucleic acid molecule, the encapsulation efficiency of the aminopropyltrimethoxysilane functionalized aluminosilicate may vary from 52 to 58%, preferably 53 to 57%, more preferably 54 to 56%, and yet more preferably about 55% based on an initial amount of mRNA. In some embodiments, the MERS-CoV vaccine comprises mRNA as the nucleic acid molecule and the nucleic acid is partially encapsulated by the aminopropyltrimethoxysilane functionalized aluminosilicate. In some embodiments, the MERS-CoV vaccine comprises mRNA as the nucleic acid molecule and the nucleic acid is at least 50%, preferably at least 60%, preferably at least 70%, preferably at least 80%, more preferably at least 90%, and yet more preferably at least 95% of the nucleic acid is covered by the aminopropyltrimethoxysilane functionalized aluminosilicate.

In certain embodiments, the vaccine comprises pDNA as the nucleic acid molecule and the amount of pDNA may vary from 80 to 120 μg. In an embodiment, the vaccine may comprise 85 to 115 μg, preferably 90 to 110 μg, more preferably 95 to 105 μg, and yet more preferably about 100 μg of pDNA. In other embodiments, the vaccine comprises mRNA as the nucleic acid molecule and the mRNA may be present in the vaccine in an amount of 20 to 40 μg. In some embodiments, the mRNA may be present in the vaccine in an amount of 21 to 39 μg, preferably 22 to 38 μg, preferably 23 to 37 μg, preferably 24 to 36 μg, preferably 25 to 35 μg, preferably 26 to 34 μg, preferably 27 to 33 μg, preferably 28 to 32 μg, more preferably 29 to 31 μg, and yet more preferably about 30 μg.

Also disclosed herein are the vaccine compositions comprising the nanoparticle fusion of the present disclosure and pharmaceutically acceptable excipients. Accordingly, the vaccine compositions comprise nucleic acid sequences, their variants or fragments encapsulated with APTMOS functionalized hierarchical aluminosilicate (APTMOS-HAS) along with excipients that may include one or more lipids, salts, stabilizers, and sugars. The nucleic acid nanoparticles in the vaccine may combine with lipids to form liposomes, lipid-based molecules, and/or lipid-based nanoparticles. Cationic or non-cationic lipids may be used in the vaccines. Suitable cationic and non-cationic lipids may be those described in the prior art, for example, ionizable lipids, polyethylene glycol lipids, and/or polyamide oligomers, and the like. Salts including calcium phosphates, aluminum salts including aluminum hydroxide, aluminum phosphate, sodium salts, and/or potassium salts, and the like may be added to the vaccine for faster and more efficient delivery of the vaccines. The vaccines may further comprise stabilizers like gelatin, polysorbates, preservatives, like thimerosal, and the likes. Sugars, including sucrose, glucose, and/or lactose, and the like, may also be added to the vaccines as they act as preservatives. The vaccine composition comprising the nanoparticle fusion of the present disclosure may comprise any vaccine additives known in the art.

The MERS-CoV vaccine may be formulated as a liquid, semi-solid, solid, or suspension, depending on the route of administration. Any suitable route for the administration of the vaccine may be chosen. The MERS-CoV vaccine may be administered to animal models, including human subjects. For example, the vaccine may be delivered via injection through intramuscular, intradermal, or subcutaneous routes, and accordingly, an injectable formulation may be prepared. In the case of administration by injection, the vaccine can be delivered with or without using a needle. In a preferred embodiment, the MERS-CoV vaccine is injected intramuscularly via needle administrations to lower the risk of contracting MERS-CoV infection.

### EXAMPLES

The disclosure will now be illustrated with working examples intended to demonstrate the working of the disclosure and not to restrictively imply any limitations on the scope of the present disclosure. The working examples depict an example of the method of the present disclosure.

The following examples demonstrate the preparation of the MERS-CoV vaccine as described herein. The examples are provided solely for illustration and are not to be construed as limitations of the present disclosure, as many variations thereof are possible without departing from the spirit and scope of the present disclosure.

### Example 1: PDNA Synthesis for Preparation of Vaccine

The S gene sequence for MERS-CoV was retrieved from PubMed (ANF29162.1). The pDNA vaccine was constructed bioinformatically using a codon selection approach. Negative cis-acting sites (splice sites, TATA boxes, etc.) that can negatively affect the gene expression were eliminated wherever possible. GC contents were adjusted to prolong the half-life of mRNA. Codon selections were adapted to Homo Sapiens. NheI and BamHI restriction sites were added upstream and downstream of the synthesized sequence, respectively. Kozac sequence and a stop codon were added upstream and downstream of the optimized S gene sequence, respectively. In total, a sequence total of 4059 nucleotides was synthesized.

The vaccine was de novo synthesized (GenArt, Germany). The S.opt.FL gene insert was cloned into pVAX1. The nucleotide sequence was confirmed by gene sequencing. The construct was transformed into HB101-competent cells and incubated in LB media with kanamycin at 37° C. overnight. For pDNA production, a successfully cloned pDNA vaccine construct was grown in LB media containing kanamycin and incubated overnight at 37° C. A ChargeSwitch-Pro filter plasmid Maxiprep purification kit (Cat #CS31106, Thermo Fisher Scientific, USA) was used to purify the S.opt.FL pDNA.

### Example 2: MRNA Synthesis for Preparation of Vaccine

For the mRNA vaccine, the mRNA was synthesized in-vitro using mMessege mMachine T7 kit (Cat #AM1344, Thermo Fisher Scientific, Lithuania). Briefly, a linearized DNA from the S.opt.FL pDNA containing T7 promoter was prepared. The transcribed and capped mRNA was synthesized according to the manufacturer's protocol. Polyadenylation was carried out using a PolyA tailing kit (Cat #AM1350, Thermo Fisher Scientific, Lithuania) according to the manufacturer's recommendation (polyA tailing (300 nucleotides)). mRNA purification was carried out using a MEGAclear transcription clean-up kit (Cat #AM1908, Thermo Fisher Scientific, Lithuania) was used to remove the remaining DNase, the nucleotides, short oligonucleotides, proteins, and salts from RNA. The quality and integrity of mRNA were assessed using 1% agarose TAE gel electrophoresis and visualized under ultraviolet (UV) light using 1 μg/mL ethidium bromide.

### Example 3: Synthesis of Silane Functionalized Hierarchical Aluminosilicate

Monodispersed spherical silica (silica) was used (Superior silica, USA). Hierarchical Aluminosilicate (HAS) (SiO₂/Al₂O₃ ratio 80) with micro and meso hexagonal pores was synthesized through a top-down approach using nanozeolitic seed and mesoporous template CTAB [Ravinayagam, V. and Jermy, B. R., Fabricating hierarchical ZSM-5 to induct long chain antioxidant coenzyme Q10 for biomedical application. Journal of Saudi Chemical Society, 2019, 23 (4), pp. 461-476, which is incorporated herein by reference in its entirety]. The parent ZSM-5 (zeolite) was dissolved in an alkaline solution in the presence of a template and then pH adjusted using dilute sulphuric acid. The material was left for hydrothermal aging for a few days and then collected through filtration, washing, drying, and calcination steps. The obtained nanomaterial was further modified by refluxing with 3-aminopropyltrimethoxysilane in a toluene solution. After silane addition, the solution mixture was refluxed for 6 hours under an argon atmosphere. Then, the solution was centrifuged, and the precipitated sample was washed and dried. The sample was denoted as APTMOS-HAS. A similar refluxing procedure was carried out for the other sample, including silica, and denoted as APTMOS-silica.

### Example 4: Characterization

The functionalized nanosupport was extensively characterized using various physicochemical techniques. The phase of hierarchical mesoporous material was analyzed using X-ray diffraction (XRD). The textural characters (such as surface area and porosity) were measured by nitrogen adsorption technique (at 77 K using an ASAP-2010 porosimeter, Micromeritics Corporation, GA). The morphology of functionalized aluminosilicate was evaluated through the electron microscopy technique (HRTEM). The silanol surface of silica was observed using Mid-infrared spectra on a Nicolet (Avatar 360) and UV-Vis spectrophotometer instrument. Thermogravimetric-differential thermal analysis (TG-DTA) was used to study the amination content. The morphology of HAS and APTMOS-HAS samples were analyzed using transmission electron microscopy (JEM2100F from JEOL Ltd, Tokyo, Japan).

### Example 5: pDNA and mRNA Encapsulation

A molecular weight of 1.5 mg with APTMOS-HAS was mixed with 100 μl of TE-buffer (pH 8.1) and, using a pipette, it was homogenized. Then, DNA or mRNA of 100 μg was added to each tube and vortexed at 22° C. for 24 hours. An evaluation of encapsulation efficiency was determined by calculating the level of pDNA and mRNA in the supernatant. The encapsulation efficiency was calculated as follows: (Amount of pDNA or mRNA present in aluminosilicate/Initial amount of pDNA or mRNA taken)×100. To evaluate encapsulation stability, heat inactivation at 95° C. for an hour and endonucleases inactivation at 37° C. for 20 minutes, followed by enzyme deactivation at 70° C. for 30 minutes was performed.

### Example 6: Animal Immunizations

6 to 8-week-old male B57BL/6J were kindly provided by King Faisal Specialist Hospital and Research Center. The mice were randomly assigned to their respective group and were housed and treated at the animal house facility at Imam Abdulrahman Bin Faisal University. Mice were assigned 6 per group. A total of 5 groups received either of the following: pDNA, mRNA, pDNA+nanosilica, mRNA+Nanosilica, or control. Mice were immunized with 100 μg pDNA in a total volume of 200 μL PBS delivered intramuscularly into the tibialis anterior muscle with a needle. Mice were immunized at days 0 and 14. Blood was collected from each mouse before the first immunization and 2 weeks after each immunization. Serum from all mice groups were collected at week 6 for immunogenicity testing, and mice were subsequently sacrificed.

### Example 7: ELISA

96-well plates (Cat #442404Thermos Fisher, Denmark) were coated with 1 μg/mL of the full-length S antigen (Cat #40069-V08B, Sinobiologic, China), S1 antigen (Cat #40069-V08H, Sinobiologic, China) of MERS-CoV or full-length S antigen (Cat #40069-V08B, Sinobiologic, China) or S antigen (Cat #442404, Thermo Fisher, Denmark) of SARS-CoV-2 and incubated for 1 hour at room temperature. Plates were washed five times with 300 μl of 1×PBS. 200 μl of a blocking reagent of 5% non-fat dry milk (Cat #170-6404, Bio-Rad, USA) in tris buffer was added to each well and incubated for 1 hour at room temperature. After 5 washes, 100 μl of serially diluted serum from the vaccinated mice was added to each well and incubated for 1 hour at room temperature. After five washes with 300 μl of 1×PBS, 100 μl if rabbit anti-mouse IgG secondary antibody conjugated with horseradish peroxidase (HRP) (Cat #31430, Invitrogen, USA) was added to each well and incubated for 1 hour at room temperature. Plates were washed five times with 300 μL of 1×PBS, and 3,3′,5,5′ TMB substrate (Cat #34021, Thermo Fisher Scientific) was added to each well according to the manufacturer's instructions. 100 μl of 2 M of sulfuric acid (2 M H₂SO₄) was added to all wells to stop the reactions. Lastly, absorbance was determined at an optical density (OD) value at 450 nm by a microplate reader (DIAsource). The end-point titer was calculated as reciprocal to the highest serum dilution when the OD value was 2 folds higher than the pre-bleed serum.

### Results

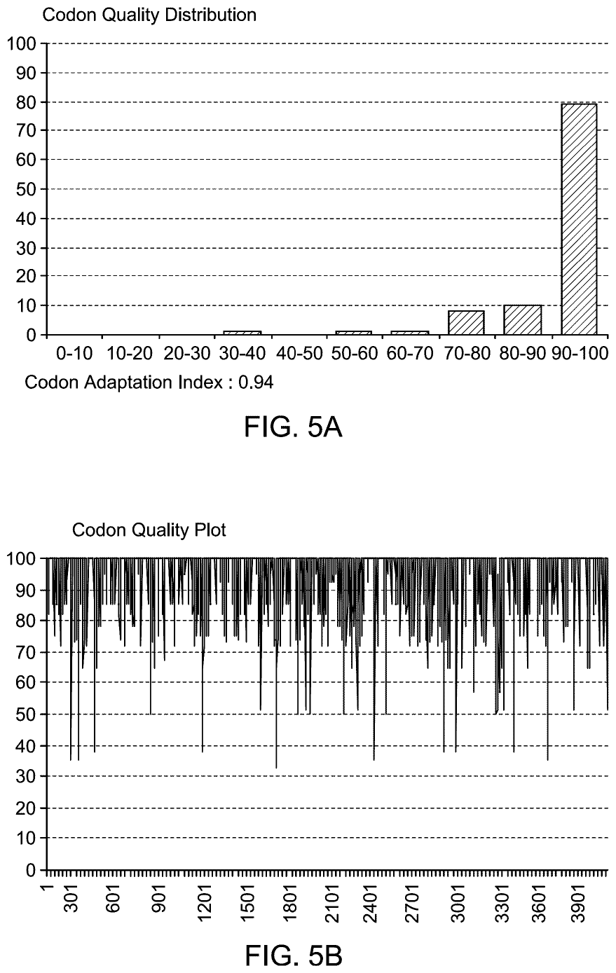

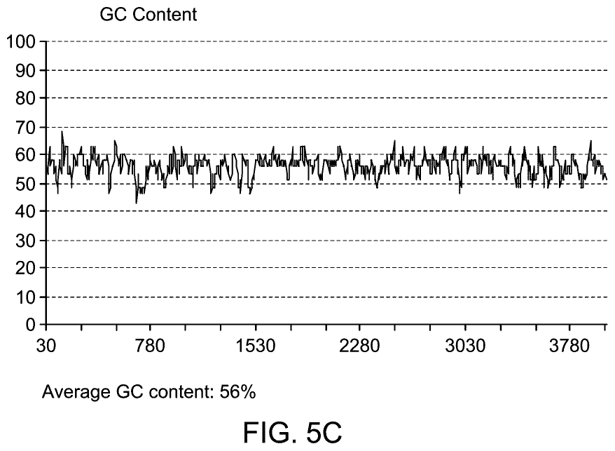

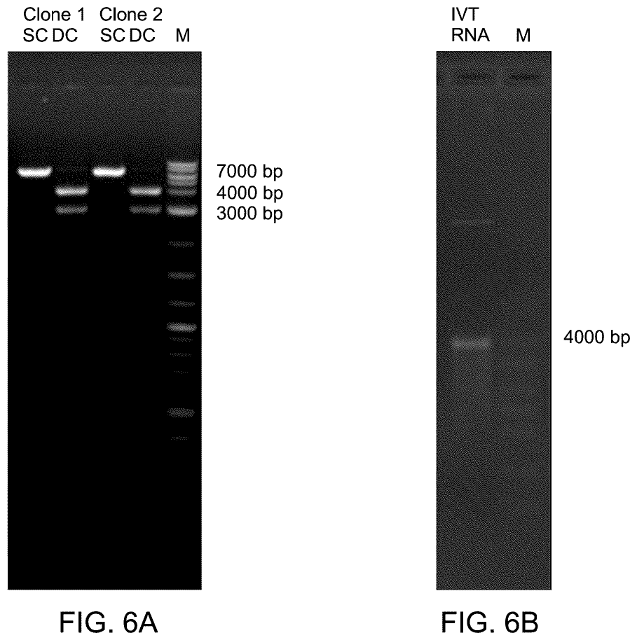

No previous study had explored the mRNA vaccine as a potential candidate against MERS-CoV. While surface glycoprotein for MERS-CoV is immunogenic, in-vitro synthesis of mRNA encoding S can be challenging. The sequence length of the S gene is 4059 bp. Therefore, optimization of gene sequence is done before pDNA and mRNA synthesis. To develop MERS-CoV nucleic acid vaccine candidates, the S gene was de-novo synthesized. S gene sequence (2016 strain) was codon optimized for mammalian codon preference (Homo Sapiens) to enhance gene expression upon immunization. During the synthesis of the codon-optimized S gene, the following steps were avoided: internal TATA boxes, chi-sites, and ribosomal entry sites, and AT-rich or CG-rich sequence stretches. Furthermore, to avoid premature termination of codon-optimized DNA during mRNA synthesis, the following were avoided: RNA instability motif repeats, RNA secondary structures, and cryptic splice donor and acceptor sites in higher eukaryotes. GC contents are adjusted to prolong mRNA half-life, and PolyA adenylation is added after mRNA synthesis of long S transcript (FIGS. 5A-5C). FIGS. 5A-5C show codon quality of MERS-CoV S gene obtained by GeneOptimizer. FIG. 5A and FIG. 5B show histograms demonstrating the percentage of sequence codons that fall within selected optimization parameters. The quality value of the most frequently used codons for given amino acids in Homo sapiens is set to 100. The plots in FIG. 5A and FIG. 5B show the quality of codons at the indicated codon position. FIG. 5C shows the average GC contents in 40 bp window centered at the indicated nucleotide position with an average GC content equal to 56%. The optimized S gene sequence was then chemically synthesized (˜4059 nucleotides) and cloned into pVax1 vector at BamhI and NheI flanking sites. The successfully cloned pDNA encoding S gene is purified, and correct band sizes are determined by BamHI and NheI (FIG. 6A). FIG. 6A shows agarose gel electrophoresis results for the synthesized of pDNA vaccine encoding S gene of MERS-CoV. Two clones of pDNA expressing S gene are seen in FIG. 6A. A correct band size of S gene 4000 bp and pVax1 (3000) is shown in the gel double cut (DC) restriction analysis of BamHI and NheI.

To synthesize the mRNA vaccine, the circular S pDNA was linearized by BamHI. The linearized S pDNA serves as a template for mRNA synthesis. As such, in-vitro synthesis using T7 polymerase, starting at the T7 promoter and terminating at the BamHI site, was conducted. Purification and evaluation of the correct mRNA synthesis was visualized by gel electrophoresis (FIG. 6B). FIG. 6B shows in-vitro (IVT) synthesized RNA at a correct band size of 4000 bp, in parallel with the RNA marker (M).

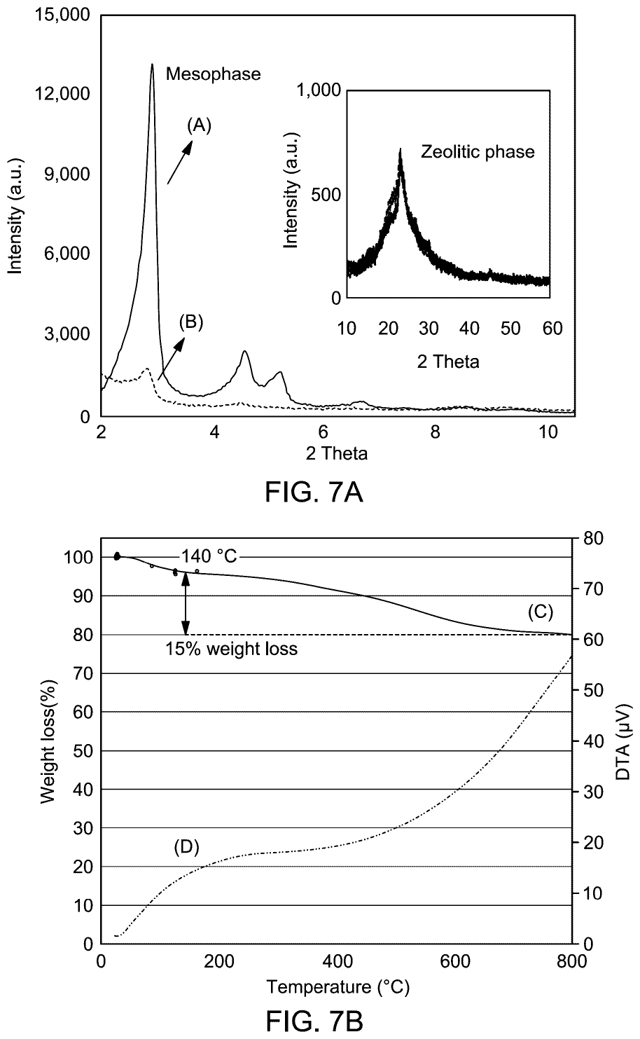

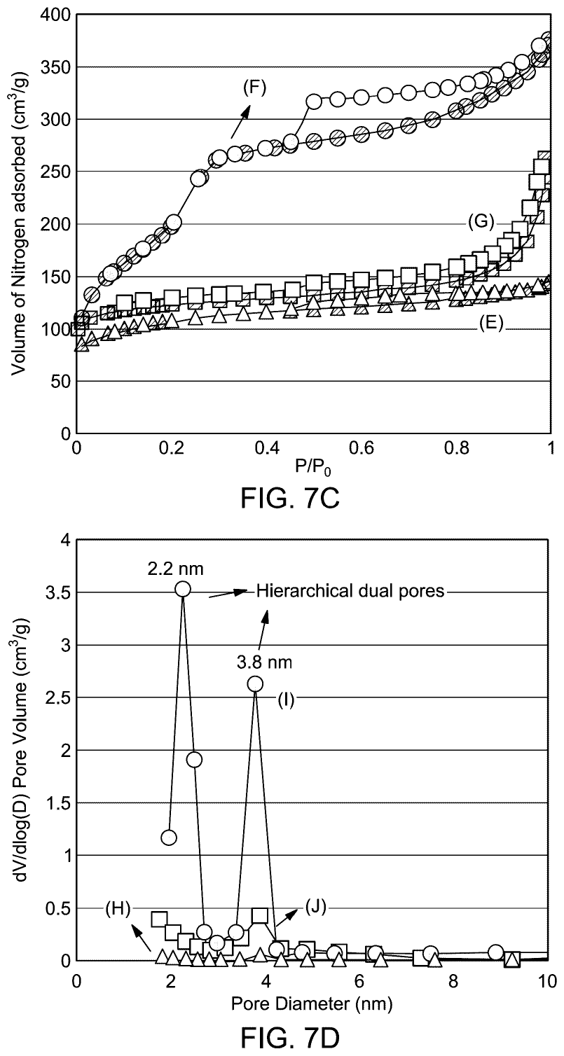

The formation of mesophase and microphase of HAS and APTMOS-HAS was confirmed using X-ray diffraction analysis (FIG. 7A). HAS ((A) in FIG. 7A) exhibited high-intensity hexagonal structured peaks, indicating the presence of (100), (110), and (200) planes of ordered mesoporous MCM-41 structure. For APTMOS-HAS ((B) in FIG. 7B), the mesophase peak intensity decreases (8-fold), while the zeolitic phase remains constant with APTMOS functionalization (see insert of FIG. 7A), indicating the silane functionalization occupies mostly on the mesophase. Thermogravimetric analysis of APTMOS functionalized on HAS showed a four-step disintegration pattern (FIG. 7B). An initial weight loss up to 140° C. is attributed to the water removal. The disintegration up to 300° C. corresponds to the amino group of silane. Further decomposition up to 600° C. is attributed to the chain breaking of silane chain moiety. Subsequently, the fourth step up to 800° C. is assigned to the silanol group disintegration. The overall weight loss for APTMOS-HAS was 20%, while the APTMOS corresponds to about 15%.

FIG. 7C shows the nitrogen adsorption isotherm of ZSM-5 (E), HAS (F), and APTMOS-HAS (G), and FIG. 7D shows the pore size distribution of ZSM-5 (H), HAS (I), and APTMOS-HAS (J). The ZSM-5 shows the micropore filling effect with lower nitrogen uptake typical of the MFI structure type. Microporous ZSM-5 showed a lower surface area of 400 m²/g with a pore volume of 0.25 cm³/g. For HAS, an intermediate type IV and I isotherm pattern was observed, indicating the presence of a hierarchical type of pore (micropores/mesopores). The mesoporous surface area increases to 846 m²/g with a pore volume of 0.58 cm³/g. The presence of dual types of pores was obvious, with pore size distribution at 2.2 nm and 3.8 nm (F and I). The surface area of APTMOS functionalized HAS reduces the surface area to about 138 m²/g with a pore volume of 0.28 cm³/g. The average pore size increases to about 8.2 nm. A reduction in pore volume (48%) and a subsequent increase in pore size from 3.8 nm to 8.2 nm indicates the effect of silylation formation by APTMOS at the external porous surface of hierarchical HAS (G and J).

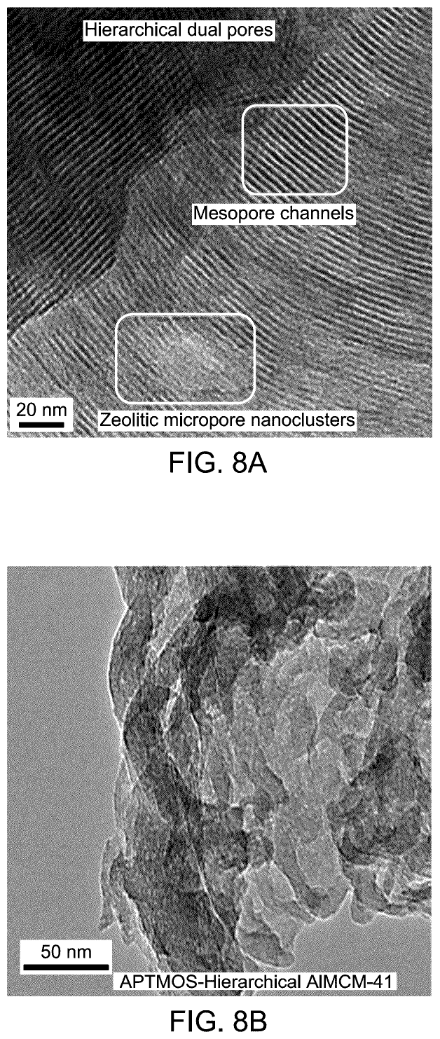

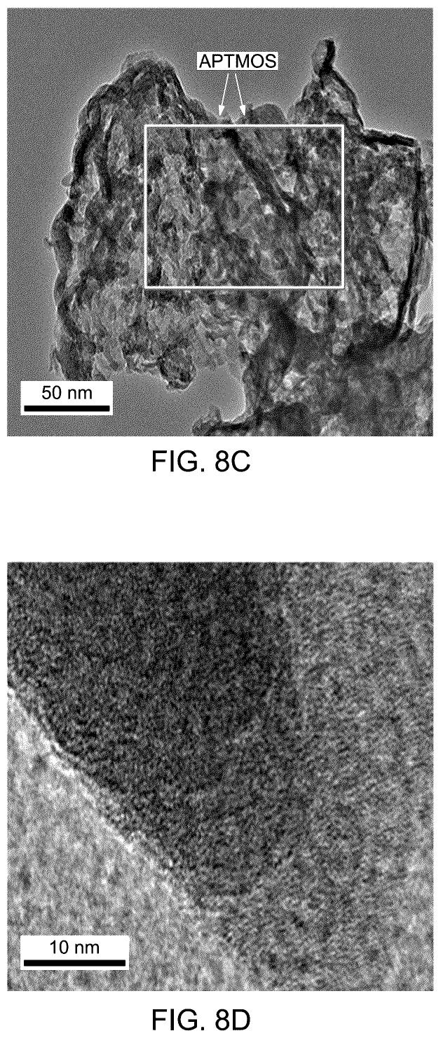

FIG. 8A to FIG. 8D shows the morphology of HAS and APTMOS-HAS. For HAS, the presence of hierarchical dual pores of micropore and mesopore channels can be seen, indicating the characteristics of zeolitic micropore nanoclusters and hexagonal MCM-41. For APTMOS-HAS, a thick formation occurs on the pores of HAS after silane functionalization, reflecting a distinct change on the external surface of hierarchical HAS (FIG. 8B-FIG. 8D). These changes of pore ordering correlate with the x-ray diffraction and surface area measurement (FIG. 7A, FIG. 7C, and FIG. 7D) further confirming a textural modification with APTMOS. A large surface area of about 138 m²/g (Table 1), even after silylation, shows that HAS can be dispersed in TE buffer for pDNA and mRNA encapsulation, as shown in the present invention.

TABLE 1
Textural Characteristics of ZSM-5, HAS, and APTMOS-HAS.

| | | | BJH | t-plot | | | |
|---|---|---|---|---|---|---|---|
| | | BET | adsorption | external | | Pore | |
| | | Surface | cumulative | surface | Pore | diameter | |
| | SiO₂/Al₂O₃ | area | surface area | area | volume | (desorption) | |
| Sample | ratio | (m²/g) | (m²/g) | (m²/g) | (cm³/g) | (nm) | |
| ZSM-5 | 80 | 400 | 137 | 170 | 0.25 | 2.5 | — |
| HAS | 80 | 846 | 806 | 1142 | 0.58 | 2.2 | 3.8 |
| APTMOS-HAS | 80 | 138 | 84 | 114 | 0.28 | 8.2 | |

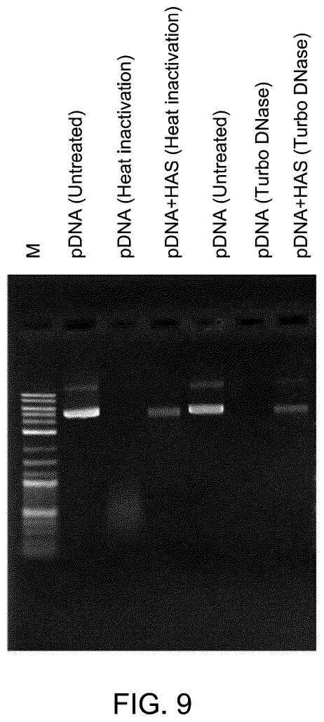

Two types of silane-functionalized nanoparticles, such as APTMOS-HAS and APTMOS-silica, were synthesized for vaccine delivery. Following encapsulation efficiency, APTMOS-HAS was determined as the best nanoparticle candidate for mRNA and pDNA, with encapsulation efficiency of 55% and 47%, respectively (Table 2). Further, it was determined that APTMOS-HAS protected the nucleic acid from degradation following heat inactivation at 96° C. after endonuclease treatment (FIG. 9).

TABLE 2
Encapsulation efficiency for the
NA MERS-CoV vaccine candidates.

| | | MERS-CoV | | |
|---|---|---|---|---|
| | | Vaccine | NA in the | |
| | MERS- | concentration | supernatant | |
| | CoV | (before | (after | Encapsulation |
| Nanocarrier | Vaccine | encapsulation) | encapsulation) | efficiency |
| APTOMS- | pDNA | 40 μg | 21 μg | 47% |
| HAS | mRNA | 40 μg | 18 μg | 55% |
| APTOMS- | pDNA | 40 μg | 13 μg | 67.5% |
| Silica | mRNA | 40 μg | 27 μg | 32.5% |

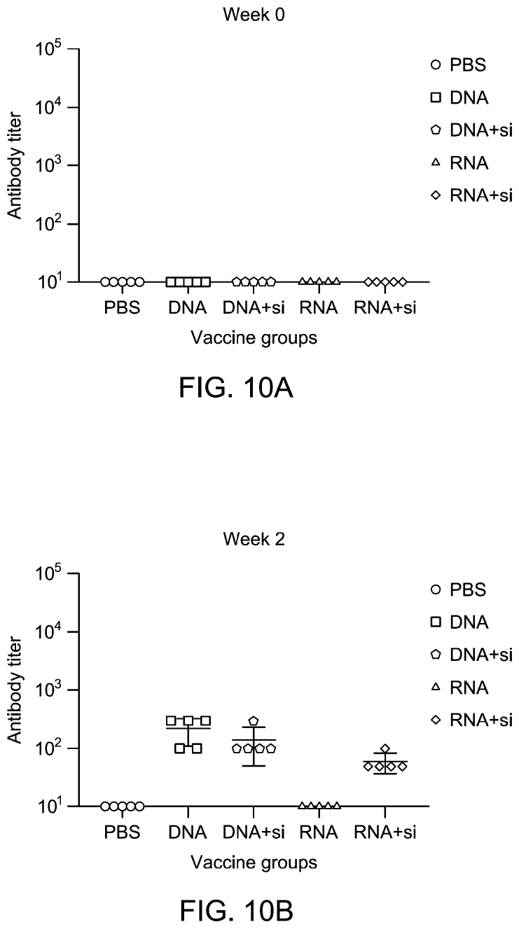

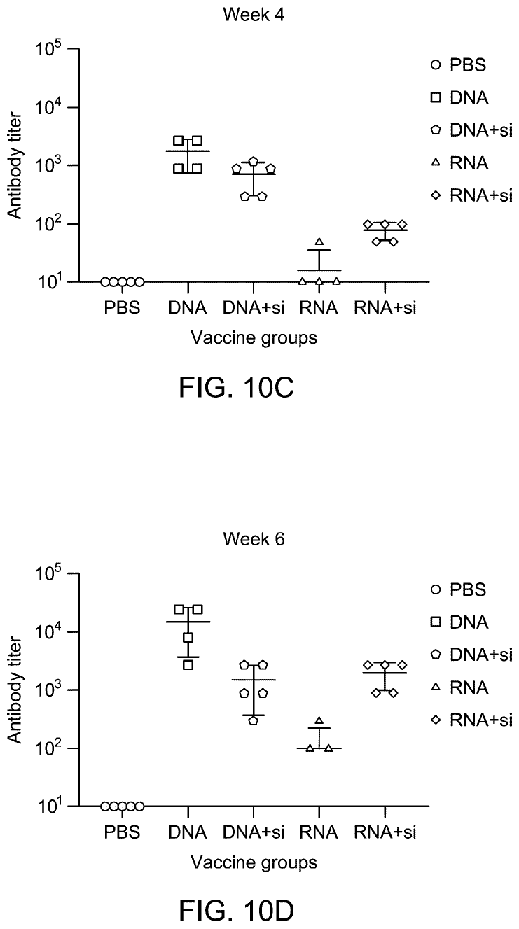

To examine the immune responses of pDNA and mRNA induced by either MERS-CoV S (pDNA, pDNA-HAS, mRNA, or mRNA-HAS), 6- to 8-week-old male B57BL/6J mice were vaccinated twice (day 0 and day 14; intramuscular (IM)) (Table 3). The humoral antibody responses in the mice group were analyzed at weeks 0, 2, 4, and 6. The sera's reactivity in immunized mice groups was measured against MERS-CoV Spike protein (S1+S2) and against the S1 domain. The pDNA vaccine elicited the highest antibody-mediated response in all mice groups, whereas mRNA elicited a lower antibody response following each immunization (FIGS. 10A-10D)

TABLE 3
Immunization schedule for the MERS-CoV vaccine candidates.

| Group | Vaccination | Dose | | Immunizations | Route |
|---|---|---|---|---|---|
| 1 | pDNA | 100 | μg | 2 | IM |
| 2 | pDNA+HAS | 100 | μg | 2 | IM |
| 3 | mRNA | 30 | μg | 2 | IM |
| 4 | mRNA+HAS | 30 | μg | 2 | IM |
| 5 | PBS | 100 | μL | 2 | IM |

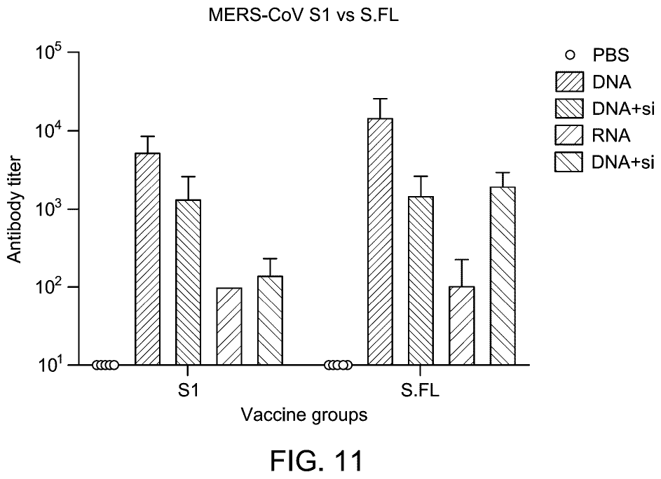

The highest antibody responses were detected at week 6, four weeks after receiving the second dose (FIG. 10D). To examine the immunogenicity after HAS encapsulation, the mRNA vaccine had slightly higher immunogenicity compared to non-encapsulated mRNA (FIG. 10B-FIG. 10D). Contrastingly, encapsulation of pDNA with HAS did not enhance the immunogenicity (FIG. 10B-FIG. 10D). The antibody titer elicited against a full-length spike (S.FL) was higher than the antibody titer generated against S1 in all vaccine groups (FIG. 11). This supports the importance of vaccinating the complete domain of MERS-CoV spike.

The present disclosure is directed to developing and testing MERS-CoV vaccines against candidates encapsulated with hierarchical aluminosilicate tailored for delivering mRNA with large molecular size. The hierarchical aluminosilicate and spherical silica with different topologies, sizes, and pore sizes were investigated. Hierarchical aluminosilicate was a better candidate for pDNA and mRNA encapsulation. A slight improvement in immunogenicity after mRNA encapsulation was observed. Humoral immune response was improved after encapsulating mRNA with hierarchical aluminosilicate. The low titer antibody response was observed in mRNA vaccine groups, suggesting further improvement and optimization of nanoparticle pore size/design.

pDNA vaccine technology is stable and is a flexible platform with a capability to induce both humoral and cellular immune responses without the need for further encapsulation steps. This is also achieved through a robust, simple, and scalable GMP production. The pDNA vaccine elicited the highest antibody response compared to mRNA alone or encapsulated mRNA. No statistically significant immunogenicity enhancement following further pDNA encapsulation is observed. Further immunogenicity characterizations to evaluate IgG subsets, neutralizing antibody response, and durability of immune response may be used for determining vaccine efficacy.

The pDNA vaccine developed against the S.FL protein of SARS-CoV-1, SARS-CoV-2, and MERS-CoV elicited high immune responses. The pDNA vaccine candidate alone elicited the highest immune response following two doses without statically significant enhancement of immunogenicity after encapsulation.

Numerous modifications and variations of the present disclosure are possible in light of the above teachings. It is therefore to be understood that within the scope of the appended claims, the disclosure may be practiced otherwise than as specifically described herein.

1: A Middle East Respiratory Syndrome coronavirus (MERS-CoV) vaccine, comprising:
a nanoparticle fusion,
wherein the nanoparticle fusion comprises an immunogenic composition and an aluminosilicate,
wherein the immunogenic composition comprises a nucleic acid molecule with a nucleic acid sequence having at least 90% identity over an entire length of the nucleic acid sequence from a spike(S) gene of a MERS-CoV strain,
wherein the nucleic acid molecule is in the form of either a plasmid deoxyribonucleic acid (pDNA) or a messenger ribonucleic acid (mRNA),
wherein the aluminosilicate is functionalized with an aminopropyltrimethoxysilane,
wherein the nucleic acid molecule is encapsulated by the aminopropyltrimethoxysilane functionalized aluminosilicate.

2: The MERS-CoV vaccine of claim 1, wherein the aminopropyltrimethoxysilane is 3-aminopropyltrimethoxysilane.

3: The MERS-CoV vaccine of claim 1, wherein the pDNA is made by a process, comprising:
cloning the nucleic acid sequence into a pVax1 vector to form a construct;
transforming the construct into HB101 competent cells;
incubating the cells in a lysogeny broth media containing kanamycin at 35 to 40° C. for 8 to 16 hours; and
purifying the incubated cells to extract the pDNA.

4: The MERS-CoV vaccine of claim 3, wherein cloning the nucleic acid sequence into the pVax1 vector includes splicing the pVax1 vector at a BamHI flanking site and a NheI flanking site.

5: The MERS-CoV vaccine of claim 1, wherein the mRNA is made by a process, comprising:
preparing a linearized DNA from the pDNA,
wherein the linearized DNA contains a T7 promoter, and
transcribing and capping the linearized DNA into the mRNA using a mMESSAGE transcription kit.

6: The MERS-CoV vaccine of claim 1, wherein a molar ratio of silicon dioxide to aluminum oxide in the aluminosilicate is from 70:1 to 90:1.

7: The MERS-CoV vaccine of claim 1, wherein the nucleic acid sequence has a length of 4000 to 4100 nucleotides.

8: The method of claim 1, wherein the nucleic acid sequence is SEQ ID No: 1.

9: The MERS-CoV vaccine of claim 1, wherein a weight ratio of the aluminosilicate to the nucleic acid sequence is from 100:1 to 200:1.

10: The MERS-CoV vaccine of claim 1, wherein in an average guanine-cytosine content (GC-content) in the nucleic acid sequence is from 40 to 70% based on a total amount of base pairs in the nucleic acid sequence.

11: The method of claim 1, wherein the aminopropyltrimethoxysilane functionalized aluminosilicate has an average pore size distribution of 8 to 9 nm.

12: The MERS-CoV vaccine of claim 1, wherein the aminopropyltrimethoxysilane functionalized aluminosilicate has a Brunauer-Emmett-Teller (BET) surface area of 120 to 160 m²/g.

13: The MERS-CoV vaccine of claim 1, wherein the aminopropyltrimethoxysilane functionalized aluminosilicate has a Barrett, Joyner, Halenda (BJH) adsorption cumulative surface area of 80 to 90 m²/g.

14: The MERS-CoV vaccine of claim 1, wherein the aminopropyltrimethoxysilane functionalized aluminosilicate has a pore volume of 0.2 to 0.4 cm³/g.

15: The MERS-CoV vaccine of claim 1, wherein the nucleic acid molecule is pDNA, and the aminopropyltrimethoxysilane functionalized aluminosilicate has an encapsulation efficiency of 45 to 50% based on an initial amount of the pDNA.

16: The MERS-CoV vaccine of claim 1, wherein the nucleic acid molecule is mRNA, and the aminopropyltrimethoxysilane functionalized aluminosilicate has an encapsulation efficiency of 52 to 58% based on an initial amount of the mRNA.

17: The MERS-CoV vaccine of claim 1, wherein the nucleic acid molecule is pDNA, and the vaccine comprises the nucleic acid molecule in an amount of 80 to 120 μg.

18: The MERS-CoV vaccine of claim 1, wherein the nucleic acid molecule is mRNA, and the vaccine comprises the nucleic acid in an amount of 20 to 40 μg.

19: A method of MERS-CoV prevention, comprising:
administering the MERS-CoV vaccine of claim 1 through one or more intramuscular needle administrations to animal models to lower a risk of contracting MERS-CoV.
